(19) United States
(12) Patent Application Publication
(10) Pub. No.: US 2026/0251632 A1
(43) Pub. Date: Aug. 27, 2026

(54) System for Predicting Physical Properties of Compressor Oil

(71) Applicant: Hitachi, Ltd., Tokyo (JP)

(72) Inventors: Shotaro YOSHITOMI, Tokyo (JP); Hayate SAITO, Tokyo (JP); Hiroshi KANEMOTO, Tokyo (JP); Shohei TERADA, Tokyo (JP)

(21) Appl. No.: 18/872,459

(22) PCT Filed: May 25, 2023
(86) PCT No.: PCT/JP2023/019570

§ 371 Date: Dec. 6, 2024

(30) Foreign Application Priority Data
Jun. 10, 2022 (JP) 2022-094634

(51) Int. Cl. G01N 33/28

(52) U.S. Cl. CPC G01N 33/2888; G01N 33/2847

(57) ABSTRACT

The present invention provides a system for predicting physical properties of compressor oil capable of predicting the degradation of compressor oil sealed in a compressor based on simple measurements and presenting an appropriate maintenance timing for compressor oil to a user. Provided is a system for predicting physical properties of compressor oil that predicts physical properties of compressor oil sealed in a compressor, the system including: a data acquisition unit that acquires measurement data representing a temporal change in pressure of a working medium of the compressor and measurement data representing a temporal change in a temperature of the working medium of the compressor; a calculation unit that calculates physical properties of the compressor oil sealed in the compressor, the physical properties including a total acid number, based on a correlation between the temporal change in the pressure of the working medium and the temporal change in the temperature of the working medium, and a temporal change in the physical properties of the compressor oil; and an output unit that outputs the calculated physical properties.

### TECHNICAL FIELD

The present invention relates to a system for predicting physical properties of compressor oil that predicts physical properties of compressor oil sealed in a compressor.

### BACKGROUND ART

In an oil-fed compressor, compressor oil is injected for lubricating a sliding unit, sealing or cooling a fluid, and other purposes. Compressor oil is generally maintained and managed by a time-based maintenance (TBM) method. When the compressor oil is used for a long period of time, oxidative degradation due to oxygen, moisture, and the like, and contamination due to friction powder, dust, and the like occur. When the compressor oil is degraded, oil film loss, sludge, and the like occur, and thus, periodic replacement using the TBM method is recommended. Conventionally, various techniques have been developed to diagnose the degradation of lubricating oils such as compressor oil.

PTL 1 describes a lubricating oil diagnosis system that acquires sensor data when a value of a degradation index based on sensor data of a chromaticity sensor is within a predetermined range and uses the sensor data for diagnosis (see claim 8 and the like). The system detects the property of the lubricating oil with various sensors including an optical sensor, determines the degree of abnormality of the lubricating oil in real time based on the sensor information), and prompts the collection of lubricating oil for detailed lubricating oil analysis at an appropriate timing according to the determination result.

PTL 2 describes a machine oil analyzer system that determines an actual remaining life for machine oil based on an ideal remaining life and a life loss factor. The ideal remaining life is determined based on a contamination factor, an initial ideal remaining life, and a temperature-based remaining life. The contamination factor is calculated based on a contaminated sample of the machine oil.

### CITATION LIST

### Patent Literature

PTL 1: JP 2021-076540 A
PTL 2: JP 2016-045198 A

### SUMMARY OF INVENTION

### Technical Problem

Conventionally, compressor oil has been maintained and managed using the TBM method, but cases have arisen where a compressor user does not observe a compressor oil replacement cycle or where the degradation of the compressor oil is overlooked. If the compressor oil is not replaced properly, an unexpected failure, such as abnormal compressor stoppage or fire, may occur. Therefore, there is a demand to visualize the degradation state and service life of compressor oil, and to establish a service solution for presenting an appropriate maintenance timing for compressor oil to a user.

In PTL 1, the degree of degradation of lubricating oil is obtained by chromaticity measured with an optical sensor. However, the change in lubricating oil chromaticity shifts to a rate-limiting stage as the degradation of the lubricating oil progresses. The change in chromaticity decreases as the degradation of the lubricating oil progresses, making it impossible to evaluate the degradation of the lubricating oil, which is problematic.

In PTL 2, a contamination factor is calculated based on a contaminated sample of machine oil. However, the contaminated sample needs to be sampled from an actual machine. The sampling method does not allow for a simple evaluation of machine oil degradation, which is problematic.

Therefore, an object of the present invention is to provide a system for predicting physical properties of compressor oil capable of predicting the degradation of compressor oil sealed in a compressor based on simple measurements and presenting an appropriate maintenance timing for compressor oil to a user.

### Solution to Problem

To solve the above problems, a system for predicting physical properties of compressor oil according to the present invention is a system for predicting physical properties of compressor oil that predicts physical properties of compressor oil sealed in a compressor, the system including: a data acquisition unit that acquires measurement data representing a temporal change in pressure of a working medium of the compressor and measurement data representing a temporal change in a temperature of the working medium of the compressor; a calculation unit that calculates physical properties of the compressor oil sealed in the compressor, the physical properties including a total acid number, based on a correlation between the temporal change in the pressure of the working medium and the temporal change in the temperature of the working medium, and a temporal change in the physical properties of the compressor oil; and an output unit that outputs the calculated physical properties.

### Advantageous Effects of Invention

According to the present invention, it is possible to provide a system for predicting physical properties of compressor oil capable of predicting the degradation of compressor oil sealed in a compressor based on simple measurements and presenting an appropriate maintenance timing for compressor oil to a user.

### BRIEF DESCRIPTION OF DRAWINGS

FIG. 1 is a diagram showing a configuration of a system for predicting physical properties of compressor oil according to a first embodiment.

FIG. 2 is a diagram showing examples of measurement results of environmental factors inside the compressor.

FIG. 3 is a diagram showing an example of a temporal change in physical properties of compressor oil.

FIG. 4 is a flowchart showing a process of predicting the physical properties of the compressor oil.

FIG. 5 is a diagram showing examples of prediction results for the physical properties of the compressor oil.

FIG. 6 is a diagram showing the relationship between the number of environmental factors used as explanatory variables and the prediction accuracy of the physical properties of the compressor oil.

FIG. 7 is a diagram showing a configuration of a system for predicting physical properties of compressor oil according to a second embodiment.

FIG. 8 is a diagram showing a relationship between the number of environmental factors used as explanatory variables and the prediction accuracy of the physical properties of the compressor oil.

FIG. 9 is a diagram showing a process example in the system for predicting physical properties of compressor oil.

FIG. 10 is a diagram showing a configuration of a system for predicting physical properties of compressor oil according to a third embodiment.

FIG. 11 is a flowchart showing a process of predicting the physical properties of the compressor oil and a process of predicting the remaining life of the compressor oil.

FIG. 12 is a diagram showing examples of prediction results for the physical properties and remaining life of the compressor oil.

FIG. 13 is a graph showing actual measurement results for the temporal change in the total acid number of the compressor oil.

FIG. 14 is a diagram showing Arrhenius plots of the actual measurement results for the temporal change in the total acid number.

### DESCRIPTION OF EMBODIMENTS

Hereinafter, a system for predicting physical properties of compressor oil according to an embodiment of the present invention will be described with reference to the drawings. In the following drawings, common components are denoted by the same reference numerals, and redundant description will be omitted.

### First Embodiment

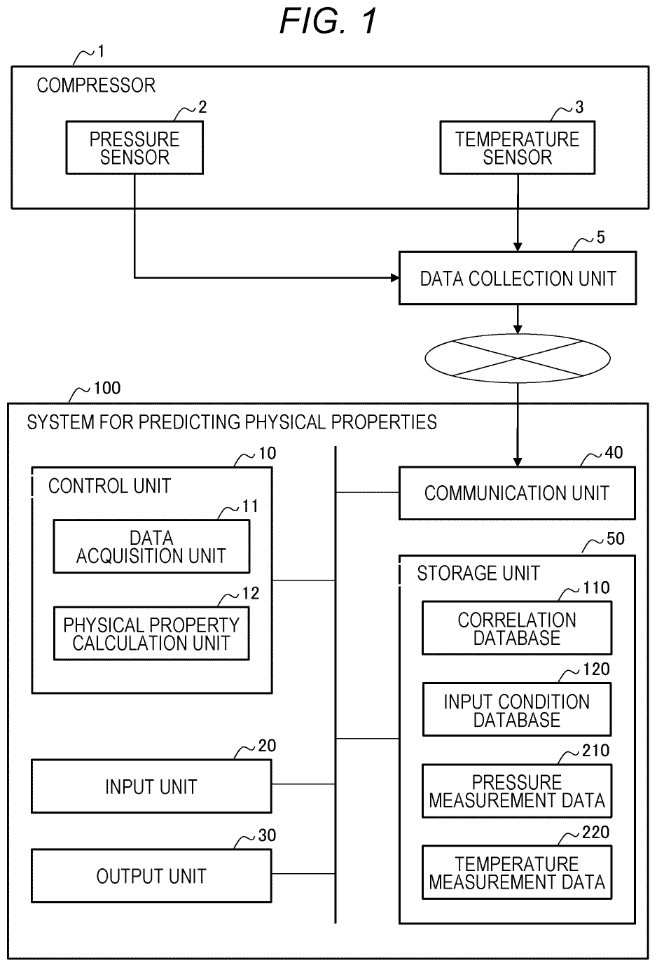

FIG. 1 is a diagram showing a configuration of a system for predicting physical properties of compressor oil according to a first embodiment.

As shown in FIG. 1, a system 100 for predicting physical properties of compressor oil according to the first embodiment includes a control unit 10, an input unit 20, an output unit 30, a communication unit 40, and a storage unit 50. The system 100 for predicting physical properties is formed of hardware such as a computer.

The system 100 for predicting physical properties is a system that predicts physical properties of compressor oil sealed in a compressor. The system 100 for predicting physical properties may predict the future physical properties of the compressor oil or may predict unknown current or past physical properties of the compressor oil. The system 100 for predicting physical properties predicts the physical properties of the compressor oil that change over time according to the degree of degradation of the compressor oil.

As the physical properties of the compressor oil, at least the total acid number of the compressor oil is predicted. The total acid number is defined as the amount of potassium hydroxide, in mg, required to neutralize acidic components contained in 1 g of a sample. The larger the total acid number, the more degraded the compressor oil.

The system 100 for predicting physical properties uses measurement results of environmental factors inside the compressor to predict the physical properties of the compressor oil. Examples of the environmental factors include the pressure of the working medium of the compressor and the temperature of the working medium of the compressor. The type of the compressor oil to be predicted and the type of the working medium to be measured are not particularly limited.

The physical properties of the compressor oil vary due to the degradation of the compressor oil and are affected by the thermal history of the compressor oil and the contact history of the compressor oil with oxygen, moisture, and the like. The thermal history depends on the temporal change in temperature inside the compressor. The contact history with oxygen, moisture, and the like depends on the temporal changes in temperature and pressure inside the compressor.

Therefore, by using the measurement results of the pressure and temperature, the physical properties of the compressor oil can be predicted from the correlation between the temporal change in the pressure of the working medium and the temporal change in the temperature of the working medium, and the temporal change in the physical properties of the compressor oil. By using the pressure and temperature of the working medium as environmental factors, measurement data on the state quantities of the environmental factors of the environment to which the compressor oil is exposed can be collected using a general measurement method.

The measurement data on the state quantities of the environmental factors used to predict the physical properties of the compressor oil is collected by a sensor installed in the compressor. By using the sensor installed in the compressor, a process of predicting the physical properties of the compressor oil can be performed based on the online measurement result without sampling the compressor oil.

As shown in FIG. 1, measurement data on state quantities of environmental factors is transmitted from a compressor 1 to the system 100 for predicting physical properties. The compressor oil to be predicted is sealed in a compression mechanism that compresses the working medium of the compressor 1, an oil tank that communicates with the compression mechanism, a heat exchanger, or the like. A pressure sensor 2 and a temperature sensor 3 are installed in the compressor 1.

As long as the compressor 1 is oil-fed, an appropriate type can be used as a prediction target. Specific examples of the compressor 1 include a screw compressor, a scroll compressor, a reciprocating compressor, a rotary compressor, and a turbo compressor. Examples of the reciprocating compressor include a piston type and a diaphragm type. Examples of the rotary compressor include a vane type and a roots type. Examples of the turbo compressor include a centrifugal type and an axial type.

The pressure sensor 2 measures the pressure of the working medium of the compressor 1. The pressure sensor 2 may measure the pressure of the mixed fluid of the working medium and the compressor oil. The pressure sensor 2 collects pressure measurement data over time. The pressure measurement data is data representing the pressure of the working medium of the compressor measured at hourly intervals. The pressure measurement data includes information on the measured value and the measurement time of the pressure.

The temperature sensor 3 measures the temperature of the working medium of the compressor 1. The temperature sensor 3 may measure the temperature of the mixed fluid of the working medium and the compressor oil. The temperature sensor 3 collects temperature measurement data over time. The temperature measurement data is data representing the temperature of the working medium of the compressor measured at hourly intervals. The temperature measurement data includes information on the measured value and the measurement time of the temperature.

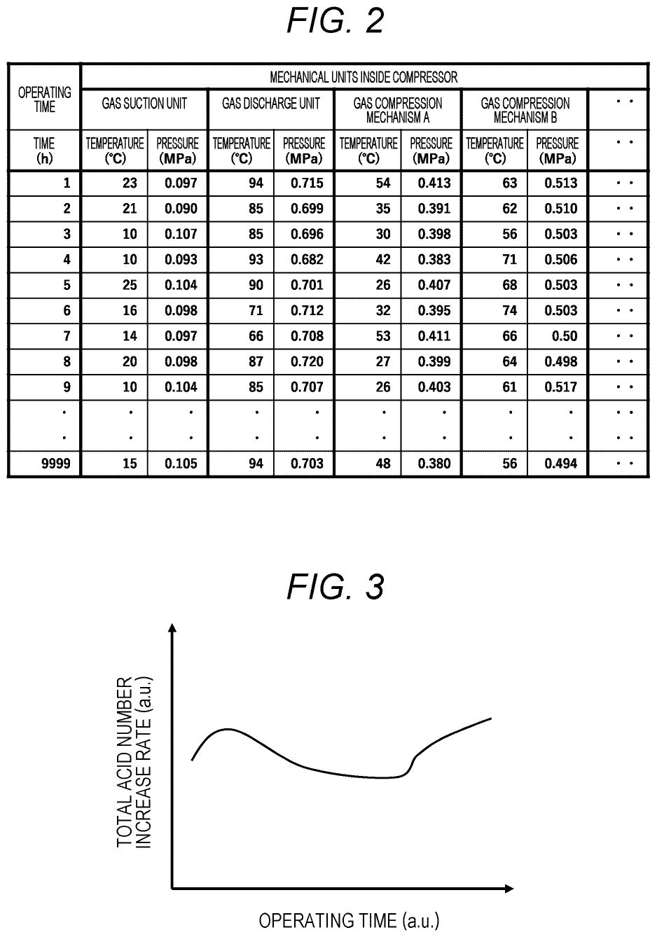

FIG. 2 is a diagram showing examples of measurement results of the environmental factors inside the compressor. FIG. 2 shows measurement results of the pressure of the working medium and measurement results of the temperature of the working medium, which were measured for each unit of the compressor.

As shown in FIG. 2, the pressure and temperature of the working medium of the compressor differ for each mechanical unit inside the compressor. The pressure and temperature of the working medium vary according to the operating time of the compressor.

The variations according to the operating time are due to changes in the environment inside the compressor. For example, the pressure and temperature measured by the sensor vary according to a change in the airtightness of the compressor, the state of the outside air, and other factors. In addition, the pressure and temperature measured by the sensor vary due to a change in physical properties caused by the degradation of the compressor oil and a change in the composition of the mixed fluid of the working medium and the compressor oil.

When the environment inside the compressor changes, in relation to the reaction of the compressor oil changing over time, the collision frequency and kinetic energy of the reaction molecules also change. Therefore, by performing multivariate regression using the temporal changes in the state quantities of the environmental factors as variables, the temporal change (change rate) in the physical properties of the compressor oil sealed in the compressor can be estimated.

In FIG. 2, the pressure and temperature of the working medium are collected individually for a gas suction unit, a gas discharge unit, a gas compression mechanism A, and a gas compression mechanism B among the mechanical units inside the compressor. The gas, which is the working medium, is sucked from the gas suction unit, compressed stepwise by the gas compression mechanism A and the gas compression mechanism B in this order, and then discharged from the gas discharge unit. The measurement data can be collected by installing a sensor in each of the mechanical units as described above.

Measurement data used to predict the physical properties of the compressor oil can be collected at an appropriate location inside the compressor. The pressure measurement data and the temperature measurement data are preferably measured at the same location. The measurement data may be collected at one location or a plurality of locations. The measurement data collected at the plurality of locations can be used to predict a single physical property by multivariate regression.

The measurement data is preferably collected at a high-temperature or high-pressure location in the mechanical unit inside the compressor and is preferably collected at least at the gas discharge unit. The high-temperature or high-pressure location is a location where the degradation of the compressor oil is likely to progress. When measurement data is collected at such a location, a significant temporal change in pressure or temperature can be detected, so that the temporal change in the physical properties of the compressor oil can be estimated with high accuracy.

In FIG. 1, the system 100 for predicting physical properties is configured to acquire measurement data collected by the compressor 1 via a communication network. The pressure sensor 2 and the temperature sensor 3 are connected to a data collection unit 5 installed in the compressor 1 via a signal line.

The data collection unit 5 is provided to be able to communicate with the system 100 for predicting physical properties. The data collection unit 5 records the measurement result for the pressure collected by the pressure sensor 2 and the measurement result for the temperature collected by the temperature sensor 3 over time during the operation of the compressor 1. The measurement data collected by the data collection unit 5 is transmitted to the system 100 for predicting physical properties via the communication network.

The communication network and the signal line may be formed of a wireless line or a wired line. Examples of the communication network include the Internet and a local area network (LAN). The data collection unit 5 may be installed for each single compressor or for each plurality of compressors. The compressor may be installed in a place where the compressor is used or may be installed in a data center or the like that manages data.

In FIG. 1, the system 100 for predicting physical properties is configured to acquire measurement data via the communication network, but may be configured to acquire measurement data from a storage medium. The system 100 for predicting physical properties may be constructed in a terminal or the like at a place where the compressor is used to perform edge computing.

The control unit 10 performs operations, such as acquiring data, reading data and programs, and running programs, to execute the process of predicting the physical properties of the compressor oil. The control unit 10 is formed of, for example, a central processing unit (CPU), a read-only memory (ROM), a random-access memory (RAM), and the like.

The input unit 20 receives input of an operator's instruction for the process or other information. The input unit 20 is formed of, for example, a keyboard, a mouse, a touch panel, or the like. The input unit 20 is connected via an input interface.

The output unit 30 outputs the result of the process of predicting the physical properties of the compressor oil, system operation information, and data content information, and the like. The output unit 30 is formed of, for example, a display device such as a liquid crystal display, an organic EL display, or a cathode ray tube. The output unit 30 is connected via an output interface.

The communication unit 40 transmits and receives data to and from an external device via the communication network. The communication unit 40 is connected via a communication interface. In FIG. 1, the communication unit 40 is configured to receive measurement data from the data collection unit 5 installed in the compressor 1.

The storage unit 50 stores data and programs. The storage unit 50 is formed of a storage medium such as a hard disk, flash memory, a readable/writable magnetic disk, or an optical disk. The storage unit 50 stores a correlation database 110, an input condition database 120, pressure measurement data 210, temperature measurement data 220, and the like.

The control unit 10 executes the process of predicting the physical properties of the compressor oil using the measurement data as input. The prediction result data is output by the execution of the process. The prediction result data is data representing the physical properties of the compressor oil predicted by the process. The prediction result data can be converted into an image and displayed by the output unit 30.

The control unit 10 includes a data acquisition unit 11, a physical property calculation unit 12, a control timer, and the like. The data acquisition unit 11 and the physical property calculation unit 12 can be read as a program or a module and implemented by the control unit 10. Alternatively, the data acquisition unit 11 and the physical property calculation unit 12 may be implemented by independent or integrated hardware modules.

In response to a request for predicting the physical properties of the compressor oil, the data acquisition unit 11 acquires measurement data on state quantities of environmental factors for each compressor oil and for each compressor. As the measurement data, data in a predetermined measurement time range can be acquired.

The physical property calculation unit 12 calculates the physical properties of the compressor oil sealed in the compressor based on the correlation between the temporal changes in the state quantities of the environmental factors and the temporal change in the physical properties of the compressor oil. The physical property calculation unit 12 performs calculation of a temporal change in the physical properties of the compressor oil based on the correlation, and calculation for the physical properties of the compressor oil at a predetermined time based on the temporal change in the physical properties of the compressor oil.

The correlation between the temporal changes in the state quantities of the environmental factors and the temporal change in the physical properties of the compressor oil is obtained by multivariate regression prior to the process of predicting the physical properties of the compressor oil. One objective variable can be explained from a plurality of explanatory variables, with the temporal changes (time derivatives) in the state quantities of the plurality of environmental factors as the explanatory variables and the temporal change (time derivative) in the physical properties of the compressor oil as then objective variable.

The temporal change (change rate) in the physical properties of the compressor oil can be obtained by applying the measurement data representing the temporal changes in the state quantities of the environmental factors to an arithmetic expression based on the correlation between the temporal changes in the state quantities of the environmental factors and the temporal change in the physical properties of the compressor oil. By time-integrating the temporal change (change rate) in the physical properties of the compressor oil, the predicted value of the physical properties of the compressor oil at any given time can be obtained.

As the arithmetic expression based on the correlation between the temporal changes in the state quantities of the environmental factors and the temporal change in the physical properties of the compressor oil, when the total acid number of the compressor oil is obtained using the pressure and temperature of the working medium as the environmental factors, an arithmetic expression represented by the following Equation (1) can be used.

$$\begin{matrix}[ Equation 1 ] & \\ a ( t ) = {f}_{pressure} ( P ( t ) ) \times {f}_{temperature} ( T ( t ) ) & ( 1 )\end{matrix}$$

[In Equation (1), a(t) represents the increase rate of the total acid number of the compressor oil at time t, P(t) represents the temporal change in the pressure of the working medium at time t, T(t) represents the temporal change in the temperature of the working medium at time t, fₚᵣₑₛₛᵤᵣₑ represents the correlation function between the pressure of the working medium and the total acid number during the change in the compressor oil over time, and fₜₑₘₚₑᵣₐₜᵤᵣₑ represents the correlation function between the temperature of the working medium and the total acid number during the change in the compressor oil over time.]

The temporal change P(t) in the pressure of the working medium at time t and the temporal change T(t) in the temperature of the working medium at time t can be calculated for an appropriate measurement time range. For example, the calculation can be performed for each minute, hour, or other hourly unit, or for each day, or other daily unit.

The time t can be based on an appropriate time. The time t may be based on any of the following: the operating time of the compressor during which the compressor compression process is performed, the usage time of the compressor calculated from the start of the use of the compressor, the usage time of the compressor oil calculated from the start of the use of the compressor oil, or other times. The time t is preferably based on the operating time of the compressor from the viewpoint of accurately predicting the change in the physical properties of the compressor oil over time due to degradation.

The correlation function fₚᵣₑₛₛᵤᵣₑ is formulated as a multivariable function that includes pressure and total acid number as variables. The correlation function fₜₑₘₚₑᵣₐₜᵤᵣₑ is formulated as a multivariable function that includes temperature and total acid number as variables. The correlation function fₚᵣₑₛₛᵤᵣₑ and the correlation function fₜₑₘₚₑᵣₐₜᵤᵣₑ may include a variable related to the sensor, a variable related to the compressor, a variable related to the working medium, and the like. Incorporation of such variables enables calibration of measurement errors, equipment errors, and the like.

The correlation function fₚᵣₑₛₛᵤᵣₑ and the correlation function fₜₑₘₚₑᵣₐₜᵤᵣₑ can be derived from a reaction rate equation based on reaction kinetics. The reaction rate equation can be an appropriate reaction order or a combination of a plurality of reaction orders different from each other, depending on the accuracy of the prediction required and other factors. Examples of elementary reactions constituting the reaction rate equation include oxidation reactions of base oil, dissociation reactions of base oil, reactions of intermediate reaction products of base oil, and the like.

The arithmetic expression based on the correlation represented by Equation (1) can be determined by multivariate analysis (fitting) using actual measurement results, with the temporal change (time derivative) in the pressure of the working medium and the temporal change (time derivative) in the temperature of the working medium as explanatory variables, and the temporal change (time derivative) in the total acid number of the compressor oil as an objective variable.

The total acid number of the compressor oil can be measured by a potentiometric titration method using a glass electrode, a silver chloride electrode, or the like, an indicator titration method using p-naphtholbenzein or the like as an indicator, or other methods. The actual measurement results for the total acid number may be collected by an accelerated degradation test by heating or the like.

FIG. 3 is a diagram showing an example of the temporal change in the physical properties of the compressor oil. FIG. 3 shows an example of the result of calculation using the arithmetic expression based on the correlation represented by Equation (1), which is the relationship between the temporal change (increase rate) in the total acid number of the compressor oil and the operating time of the compressor.

As shown in FIG. 3, the temporal change (change rate) in the physical properties of the compressor oil varies according to the pressure and temperature inside the compressor during its operation.

As the pressure and temperature of the working medium of the compressor rise, the amount of heat received by the compressor oil and the contact of oxygen, moisture, and the like with the compressor oil increase, leading to a higher increase rate of the total acid number. On the other hand, as the pressure and temperature of the working medium fall, the amount of heat received by the compressor oil and the contact of oxygen, moisture, and the like with the compressor oil decrease, leading to a lower increase rate of the total acid number.

Therefore, by measuring the temporal change in the pressure of the working medium and the temporal change in the temperature of the working medium, the temporal change (change rate) in the physical properties of the compressor oil can be obtained without actually measuring the concentration of a reactant or product during the degradation of the compressor oil. Even if the reaction order and elementary reaction of the degradation reaction of the compressor oil are unknown, it is possible to estimate the temporal change (change rate) in the physical properties of the compressor oil and the physical properties of the compressor oil at a predetermined time.

The total acid number of the compressor oil can be obtained by the following Equation (2) using the temporal change (increase rate) in the total acid number of the compressor oil.

$$\begin{matrix}[ Equation 2 ] & \\ A ( t ) = A ( 0 ) + {\int }_{0}^{{t}_{1}} a ( t ) dt & ( 2 )\end{matrix}$$

[In Equation (2), a(t) represents the temporal change (increase rate) in the total acid number of the compressor oil at time t, A(t) represents the total acid number of the compressor oil at time t, A(0) represents the total acid number of the compressor oil in the initial state, and t1 represents the elapsed time from the initial state.]

The total acid number A(0) of the compressor oil in the initial state can be obtained by actual measurement before the compressor oil is injected into the compressor. Alternatively, actual measurement can be performed at the time of sampling the compressor oil sealed in the compressor, and the time of sampling can be set to the latest initial state.

The elapsed time t1 can be designated for each request for predicting the physical properties of the compressor oil based on an appropriate time. The elapsed time t1 may be based on any of the following: the operating time of the compressor during which the compressor compression process is performed, the usage time of the compressor calculated from the start of the use of the compressor, the usage time of the compressor oil calculated from the start of the use of the compressor oil, or other times. The elapsed time t1 is preferably based on the operating time of the compressor from the viewpoint of accurately predicting the change in the physical properties of the compressor oil over time due to degradation.

The system 100 for predicting physical properties stores the correlation database 110 in advance of the process of predicting the physical properties of the compressor oil. The correlation database 110 includes correlation data representing the correlation between the temporal changes in the state quantities of the environmental factors and the temporal change in the physical properties of the compressor oil. The correlation data is prepared for each type of compressor oil and each type of compressor. The correlation data may be represented by an arithmetic expression or may be represented by a table through mapping.

The system 100 for predicting physical properties stores the input condition database 120 in advance of the process of predicting the physical properties of the compressor oil. The input condition database 120 includes input condition data representing input conditions related to the compressor oil and the compressor. The input condition data is input for each type of compressor oil or each type of compressor. The input condition data includes information on the physical properties of the compressor oil in the initial state where degradation has not progressed.

Measurement data measured by the compressor 1 is input to the system 100 for predicting physical properties in response to a request for predicting the physical properties of the compressor oil. The pressure measurement data 210 and the temperature measurement data 220 can be stored in the storage unit 50. The pressure measurement data 210 and the temperature measurement data 220 are stored as data for each request for predicting the physical properties of the compressor oil, for each type of compressor oil, or for each type of compressor.

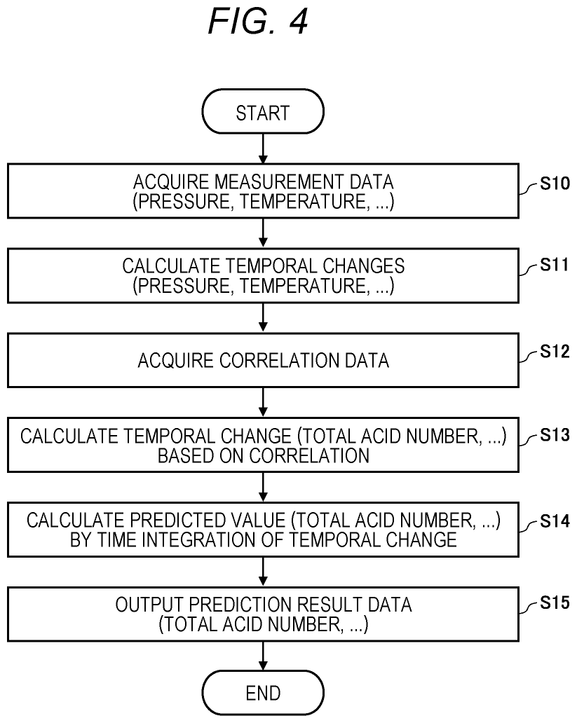

FIG. 4 is a flowchart showing the process of predicting the physical properties of the compressor oil.

As shown in FIG. 4, the process of predicting the physical properties of the compressor oil can be performed using the pressure measurement data and the temperature measurement data collected by the compressor 1 and correlation data indicating the correlation represented by Equation (1).

The control unit 10 can start the process of predicting the physical properties of the compressor oil at a designated time by an instruction from the operator. The time of prediction may be designated based on any of the following: the operating time of the compressor during which the compressor compression process is performed, the usage time of the compressor calculated from the start of the use of the compressor, the usage time of the compressor oil calculated from the start of the use of the compressor oil, or other times.

First, the control unit 10 acquires measurement data for each request for predicting the physical properties of the compressor oil (step S10). As the measurement data, data included in an appropriate measurement time range up to the latest measurement time can be read, but from the viewpoint of accurately evaluating the change of the compressor oil over time due to degradation, it is preferable to read data from the measurement time when the initial state was measured up to the latest measurement time.

Subsequently, the control unit 10 calculates the temporal change in the pressure of the working medium and the temporal change in the temperature of the working medium based on the measurement data (step S11). The temporal change is derived from a plurality of pieces of measurement data included in a predetermined measurement time range.

Subsequently, the control unit 10 acquires correlation data for each request for predicting the physical properties of the compressor oil (step S12). When the total acid number is predicted as the physical properties of the compressor oil, correlation data indicating the correlation represented by Equation (1) is read from the correlation database 110 for each type of compressor oil or each type of compressor.

Subsequently, the control unit 10 calculates the temporal change in the physical properties of the compressor oil based on the correlation between the temporal changes in the state quantities of the environmental factors and the temporal change in the physical properties of the compressor oil (step S13). When the total acid number is predicted as the physical properties of the compressor oil, the temporal change (change rate) in the total acid number of the compressor oil is derived by applying the calculated temporal changes in the pressure and temperature of the working medium to the correlation represented by Equation (1).

Subsequently, the control unit 10 time-integrates the temporal change (change rate) in the physical properties of the compressor oil to calculate the predicted value of the physical properties of the compressor oil (step S14). When the total acid number is predicted as the physical properties of the compressor oil, the total acid number of the compressor oil in the initial state and the elapsed time from the initial state are read, and the temporal change (change rate) in the total acid number of the compressor oil is time-integrated with the elapsed time from the initial state to derive the predicted value of the total acid number of the compressor oil at a designated time.

Subsequently, the control unit 10 outputs prediction result data representing the calculated physical properties of the compressor oil (step S15). The prediction result for the physical properties of the compressor oil can be output to the output unit 30 by generating an image based on the prediction result data.

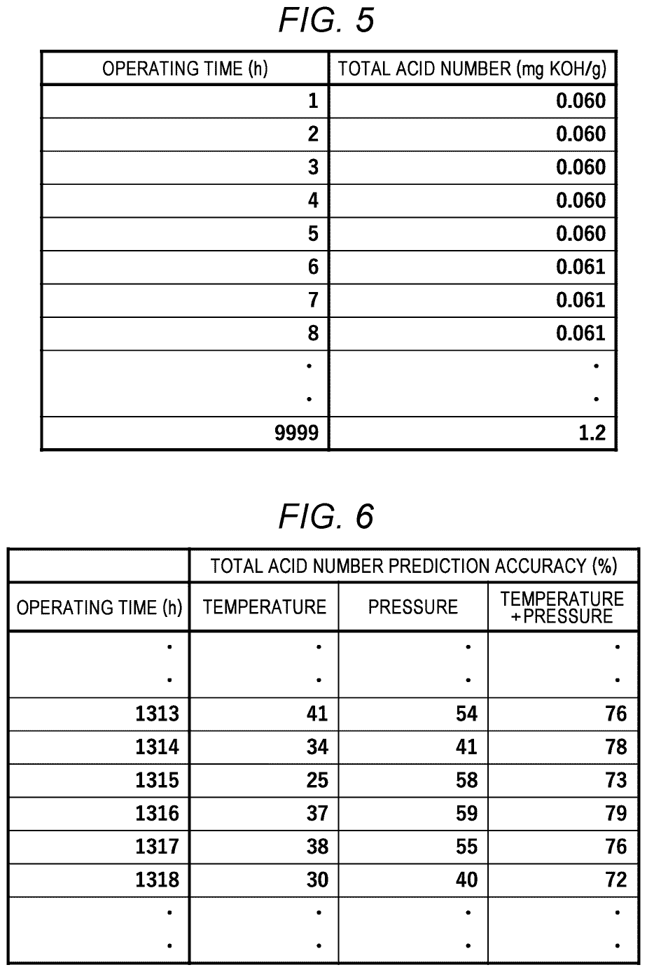

FIG. 5 is a diagram showing examples of prediction results for the physical properties of compressor oil. FIG. 5 shows the predicted value of the total acid number of the compressor oil for each operating time of the compressor, predicted as the physical properties of the compressor oil.

As shown in FIG. 5, the prediction results for the physical properties of the compressor oil can be output as time-series data associated with the elapsed time.

As the prediction result for the physical properties of the compressor oil, the predicted value of the physical properties of the compressor oil at the designated time and the predicted transition of the physical properties of the compressor oil over an appropriate time range up to the designated time can be output to the output unit 30. The predicted value of the temporal change in the physical properties of the compressor oil at the designated time and the predicted transition of the temporal change in the physical properties of the compressor oil over an appropriate time range up to the designated time may be output to the output unit 30.

In FIG. 5, the prediction result for the physical properties of the compressor oil is associated with the operating time of the compressor, but the prediction result for the physical properties of the compressor oil may be associated with the usage time of the compressor or the usage time of the compressor oil. The prediction result may be associated with a date and time. The operating time of the compressor, the usage time of the compressor, and the usage time of the compressor oil can each be converted into a date and time using an average elapsed time per day or similar method.

In FIG. 5, the prediction results for the physical properties of the compressor oil are displayed as a table, but may be displayed as a graph, a single answer, or the like. The prediction results may be displayed independently for each request for predicting the physical properties of the compressor oil or for each mechanical unit inside the compressor, or may be displayed collectively as the result of a plurality of processes. Prediction result data representing the calculated physical properties of the compressor oil can be stored in the storage unit 50 for each request for predicting the physical properties of the compressor oil.

The system 100 for predicting physical properties described above uses the correlation between the temporal change in the pressure of the working medium and the temporal change in the temperature of the working medium, and the temporal change in the physical properties of the compressor oil, to predict the physical properties of the compressor oil. Therefore, it is possible to predict the physical properties of the compressor oil due to the material amount-dependent change over time and the temperature-dependent change over time, without measuring the concentration of a reactant or product during the degradation of the compressor oil. In addition, since the measurement data collected by the sensor is used, prediction can be performed based on simple measurement. The maintenance cycle of the compressor oil is optimized, making it possible to reduce the environmental load by reducing the amount of waste oil and to prevent unexpected failures, such as abnormal stop and fire, of the compressor.

FIG. 6 is a diagram showing the relationship between the number of environmental factors used as explanatory variables and the prediction accuracy of the physical properties of the compressor oil. FIG. 6 shows results of comparing the agreement rate [%] of the predicted value of the total acid number with the measured value between the case of using only the temporal change in the temperature of the working medium, the case of using only the temporal change in the pressure of the working medium, and the case of using the temporal change in the temperature of the working medium and the temporal change in the pressure.

As shown in FIG. 6, the higher the number of environmental factors used as explanatory variables, the higher the prediction accuracy. It can be said that, to predict the physical properties of the compressor oil with high accuracy, multivariate regression based on a plurality of environmental factors is preferable.

### Second Embodiment

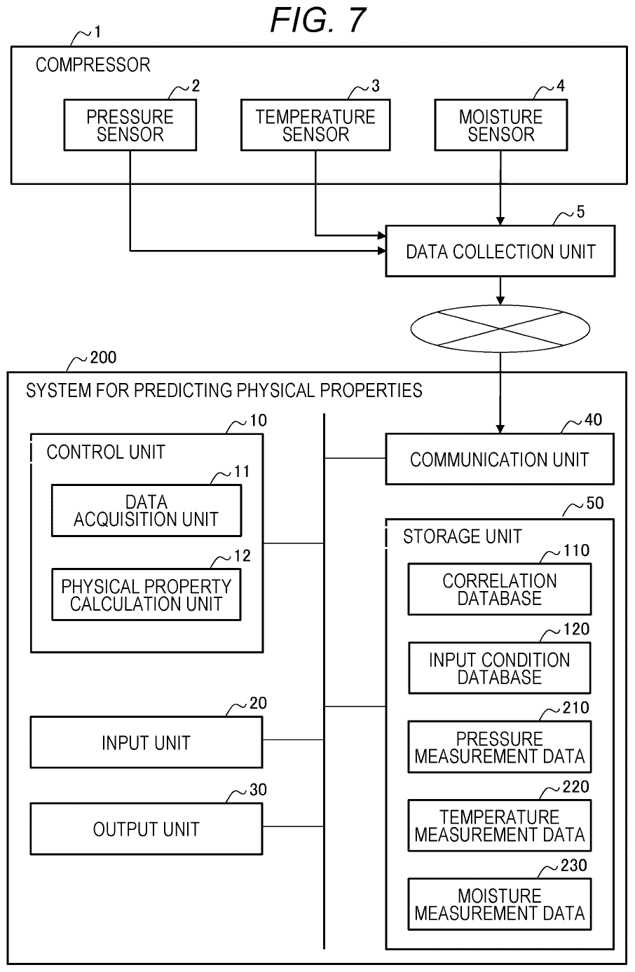

FIG. 7 is a diagram showing a configuration of a system for predicting physical properties of compressor oil according to a second embodiment.

As shown in FIG. 7, a system 200 for predicting physical properties of compressor oil according to the second embodiment includes a control unit 10, an input unit 20, an output unit 30, a communication unit 40, and a storage unit 50, similarly to the system 100 for predicting physical properties described above. The system 200 for predicting physical properties is formed of hardware such as a computer.

The system 200 for predicting physical properties is a system that predicts physical properties of compressor oil sealed in a compressor. The system 200 for predicting physical properties differs from the system 100 for predicting physical properties in that the moisture content of the working medium of the compressor is used as an environmental factor inside the compressor in addition to the pressure of the working medium of the compressor and the temperature of the working medium of the compressor. Other main configurations are similar to those of the system 100 for predicting physical properties.

The physical properties of the compressor oil vary due to the degradation of the compressor oil and are affected by the contact history of the compressor oil with moisture. The contact history with moisture depends on the temporal change in the moisture content inside the compressor. In particular, when the base oil undergoes a hydrolysis reaction as in the case of an ester-based oil or the like, water is involved in the degradation reaction of compressor oil. The reaction order and elementary reaction type of the degradation reaction are affected by the presence of water.

Therefore, by using the measurement result for the moisture content, the physical properties of the compressor oil can be predicted from the correlation between the temporal change in the pressure of the working medium, the temporal change in the temperature of the working medium, and the temporal change in the moisture content of the working medium, and the temporal change in the physical properties of the compressor oil. By using the moisture content of the working medium as the environmental factor, the degradation of the compressor oil accompanied by the hydrolysis reaction can be predicted with higher accuracy.

When the moisture content of the working medium of the compressor is used as an environmental factor, a moisture sensor 4 is installed in the compressor 1. As the moisture sensor 4, an appropriate sensor such as a capacitive sensor or a resistive sensor can be used. The moisture sensor 4 is preferably installed at the same location as the pressure sensor 2 and the temperature sensor 3.

The moisture sensor 4 measures the moisture content of the working medium of the compressor 1. The moisture sensor 4 may measure the moisture content of the mixed fluid of the working medium and the compressor oil. The moisture sensor 4 collects moisture measurement data over time. The moisture measurement data is data representing the moisture content of the working medium of the compressor measured at hourly intervals. The moisture measurement data includes information on the measured value and the measurement time of the moisture content.

In FIG. 7, the system 200 for predicting physical properties is configured to acquire measurement data collected by the compressor 1 via the communication network. The pressure sensor 2, the temperature sensor 3, and the moisture sensor 4 are connected to a data collection unit 5 installed in the compressor 1 via a signal line.

The data collection unit 5 is provided to be able to communicate with the system 200 for predicting physical properties. The data collection unit 5 records the measurement result for the pressure collected by the pressure sensor 2, the measurement result for the temperature collected by the temperature sensor 3, and the measurement result for the moisture content collected by the moisture sensor 4 over time during the operation of the compressor 1. The measurement data collected by the data collection unit 5 is transmitted to the system 200 for predicting physical properties via the communication network.

In FIG. 7, the system 200 for predicting physical properties is configured to acquire measurement data via the communication network, but may be configured to acquire measurement data from a storage medium. The system 200 for predicting physical properties may be constructed in a terminal or the like at a place where the compressor is used to perform edge computing.

The arithmetic expression based on the correlation between the temporal changes in the state quantities of the environmental factors and the temporal change in the physical properties of the compressor oil can be represented by the following Equation (3) or (4), when the total acid number of the compressor oil is obtained using the pressure, temperature, and moisture content of the working medium as environmental factors.

$$\begin{matrix}[ Equation 3 ] & \\ a ( t ) = {f}_{water} ( W ( t ) ) + {f}_{pressure} ( P ( t ) ) \times {f}_{temperature} ( T ( t ) ) & ( 3 )\end{matrix}$$

$$\begin{matrix}[ Equation 4 ] & \\ a ( t ) = {f}_{water} ( W ( t ) ) \times {f}_{pressure} ( P ( t ) ) \times {f}_{temperature} ( T ( t ) ) & ( 4 )\end{matrix}$$

[In Equations (3) and (4), a(t) represents the increase rate of the total acid number of the compressor oil at time t, W(t) represents the temporal change in the moisture content of the working medium at time t, P(t) represents the temporal change in the pressure of the working medium at time t, T(t) represents the temporal change in the temperature of the working medium at time t, f_{water} represents the correlation function between the moisture content of the working medium and the total acid number during the change in the compressor oil over time, fₚᵣₑₛₛᵤᵣₑ represents the correlation function between the pressure of the working medium and the total acid number during the change in the compressor oil over time, and fₜₑₘₚₑᵣₐₜᵤᵣₑ represents the correlation function between the temperature of the working medium and the total acid number during the change in the compressor oil over time.]

The temporal change W(t) in the moisture content of the working medium at time t can be obtained for an appropriate measurement time range. For example, the calculation can be performed for each minute, hour, or other hourly unit, or for each day, or other daily unit.

The correlation function f_{water} is formulated as a multivariable function that includes moisture content and total acid number as variables. The correlation function f_{water} may include a variable related to the sensor, a variable related to the compressor, a variable related to the working medium, and the like. Incorporation of such variables enables calibration of measurement errors, equipment errors, and the like.

The correlation function f_{water} can be derived from a reaction rate equation based on reaction kinetics. The reaction rate equation can be an appropriate reaction order or a combination of a plurality of reaction orders different from each other, depending on the accuracy of the prediction required and other factors.

The arithmetic expression based on the correlation represented by Equation (3) or (4) can be determined by multivariate analysis (fitting) using actual measurement results, with the temporal change in the pressure of the working medium (time derivative), the temporal change in the temperature of the working medium (time derivative), and the temporal change in the moisture content of the working medium (time derivative) as explanatory variables, and the temporal change in the total acid number of the compressor oil (time derivative) as an objective variable.

Similarly to the system 100 predicting physical properties described above, the system 200 for predicting physical properties stores a correlation database 110 and an input condition database 120 in advance of the process of predicting the physical properties of the compressor oil. The correlation database 110 is configured to include correlation data indicating the correlation represented by Equation (3) and correlation data indicating the correlation represented by Equation (4).

Measurement data measured by the compressor 1 is input to the system 200 for predicting physical properties in response to a request for predicting the physical properties of the compressor oil. The pressure measurement data 210, the temperature measurement data 220, and the moisture measurement data 230 can be stored in the storage unit 50. The pressure measurement data 210, the temperature measurement data 220, and the moisture measurement data 230 are stored as data for each request for predicting the physical properties of the compressor oil, for each type of compressor oil, or for each type of compressor.

The process of predicting the physical properties of the compressor oil can be performed using the pressure measurement data, the temperature measurement data, and the moisture measurement data collected by the compressor 1 and the correlation data indicating the correlation represented by Equation (3) or the correlation data indicating the correlation represented by Equation (4).

The correlation represented by Equation (3) can be used when the hydrolysis reaction can be regarded as non-pressure-dependent or non-temperature-dependent, such as when the moisture content is large and excessive. The correlation represented by Equation (4) can be used when the hydrolysis reaction can be regarded as pressure-dependent or temperature-dependent.

When the total acid number is predicted as the physical properties of the compressor oil using the pressure of the working medium of the compressor, the temperature of the working medium of the compressor, and the moisture content of the working medium of the compressor as environmental factors, measurement data is acquired (step S10), as in the process shown in FIG. 4. Then, based on the measurement data, the temporal change in the pressure of the working medium, the temporal change in the temperature of the working medium, and the temporal change in the moisture content of the working medium are calculated (step S11).

Subsequently, correlation data indicating the correlation represented by Equation (3) or the correlation represented by Equation (4) is acquired (step S12). The calculated temporal change in the pressure of the working medium, the temporal change in the pressure of the working medium, and the temporal change in the pressure of the working medium are applied to the correlation represented by Equation (3) or the correlation represented by Equation (4) to calculate the temporal change in the physical properties of the compressor oil (step S13).

Subsequently, input condition data representing the physical properties of the compressor oil in the initial state is read, and the calculated temporal change in the physical properties of the compressor oil is time-integrated to calculate the predicted value of the physical properties of the compressor oil (step S14). Thereafter, the data on the calculated prediction result for the physical properties of the compressor oil is output (step S15).

The system 200 for predicting physical properties described above uses the correlation between the temporal change in the pressure of the working medium, the temporal change in the temperature of the working medium, and the temporal change in the moisture content of the working medium, and the temporal change in the physical properties of the compressor oil, to predict the physical properties of the compressor oil. Therefore, it is possible to predict the physical properties of the compressor oil due to the material amount-dependent change over time, the temperature-dependent change over time, and the moisture content-dependent change over time, without measuring the concentration of a reactant or product during the degradation of the compressor oil. In particular, when the base oil undergoes a hydrolysis reaction as in the case of an ester-based oil or the like, highly accurate prediction can be performed.

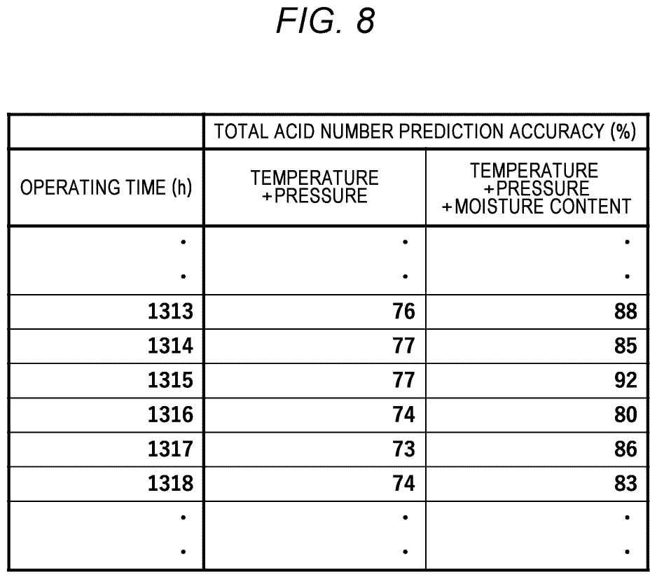

FIG. 8 is a diagram showing the relationship between the number of environmental factors used as explanatory variables and the prediction accuracy of the physical properties of the compressor oil. FIG. 8 shows a result of comparing the agreement rate [%] of the predicted value of the total acid number with the actual measured value between the case of using the temporal change in the temperature of the working medium and the temporal change in the pressure and the case of using the temporal change in the temperature of the working medium, the temporal change in the pressure, and the temporal change in the moisture content.

As shown in FIG. 8, when the moisture content was added as an environmental factor used as an explanatory variable, high prediction accuracy was obtained. It can be said that when the base oil undergoes a hydrolysis reaction, multivariate regression based on a plurality of environmental factors, including moisture content, is preferable.

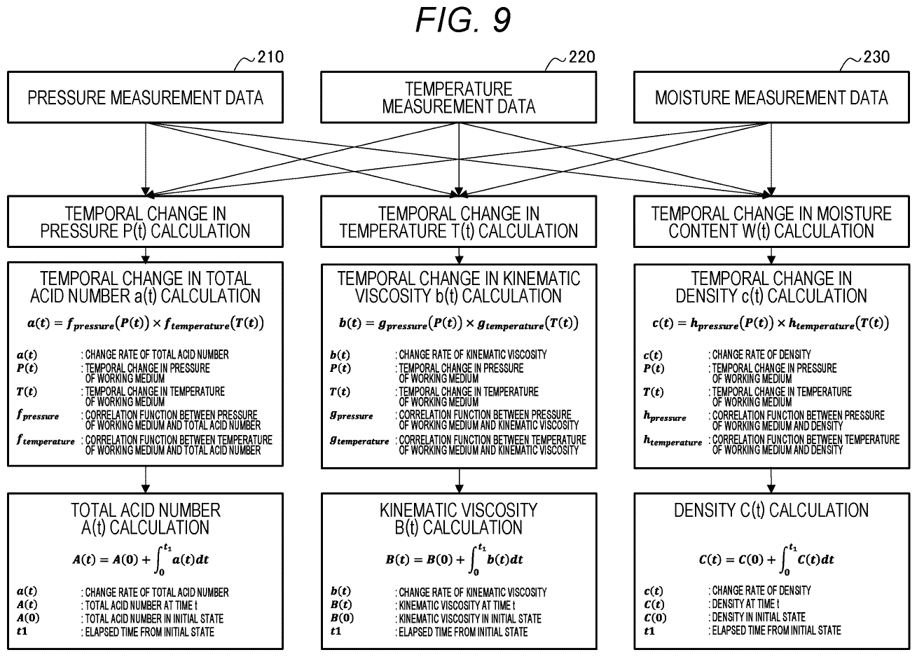

FIG. 9 is a diagram showing a process example in the system for predicting physical properties of compressor oil.

As shown in FIG. 9, the process of predicting the physical properties of the compressor may be performed in parallel for a plurality of physical properties of the compressor. In the system for predicting physical properties, as the physical properties of the compressor oil, in addition to the total acid number of the compressor oil, either the kinematic viscosity of the compressor oil or the density of the compressor oil, or both, can be predicted.

By obtaining the kinematic viscosity and the density of the compressor oil, the viscosity (viscosity coefficient) of the compressor oil can be calculated. Estimating the changes in kinematic viscosity and viscosity due to the degradation of the compressor oil makes it possible to diagnose the lubricating action of the sliding unit with the compressor oil, the sealing action of the fluid, the cooling action of the fluid, the energy efficiency of the compressor, and the like.

As the arithmetic expression based on the correlation between the temporal changes in the state quantities of the environmental factors and the temporal change in the physical properties of the compressor oil, when the kinematic viscosity of the compressor oil is obtained using the pressure and temperature of the working medium as the environmental factors, an arithmetic expression represented by the following Equation (5) can be used.

$$\begin{matrix}[ Equation 5 ] & \\ b ( t ) = {g}_{pressure} ( P ( t ) ) \times {g}_{temperature} ( T ( t ) ) & ( 5 )\end{matrix}$$

[In Equation (5), b(t) represents the change rate of the kinematic viscosity of the compressor oil at time t, P(t) represents the temporal change in the pressure of the working medium at time t, T(t) represents the temporal change in the temperature of the working medium at time t, gₚᵣₑₛₛᵤᵣₑ represents the correlation function between the pressure of the working medium and the kinematic viscosity during the change in the compressor oil over time, and gₜₑₘₚₑᵣₐₜᵤᵣₑ represents the correlation function between the temperature of the working medium and the kinematic viscosity during the change in the compressor oil over time.]

The correlation function pressure is formulated as a multivariable function that includes pressure and kinematic viscosity as variables. In addition, the correlation function gₜₑₘₚₑᵣₐₜᵤᵣₑ is formulated as a multivariable function that includes temperature and kinematic viscosity as variables. The correlation function gₚᵣₑₛₛᵤᵣₑ and the correlation function gₜₑₘₚₑᵣₐₜᵤᵣₑ may include a variable related to the sensor, variable related to the compressor, a variable related to the working medium, and the like. Incorporation of such variables enables calibration of measurement errors, equipment errors, and the like.

The correlation function gₚᵣₑₛₛᵤᵣₑ and the correlation function gₜₑₘₚₑᵣₐₜᵤᵣₑ can be derived from a reaction rate equation based on reaction kinetics. The reaction rate equation can be an appropriate reaction order or a combination of a plurality of reaction orders different from each other, depending on the accuracy of the prediction required and other factors. Examples of elementary reactions constituting the reaction rate equation include oxidation reactions of base oil, dissociation reactions of base oil, reactions of intermediate reaction products of base oil, and the like.

The arithmetic expression based on the correlation represented by Equation (5) can be determined by multivariate analysis (fitting) using actual measurement results, with the temporal change (time derivative) in the pressure of the working medium and the temporal change (time derivative)) in the temperature of the working medium as explanatory variables, and the temporal change (time derivative) in the kinematic viscosity of the compressor oil as an objective variable.

The kinematic viscosity of the compressor oil can be measured using a capillary viscometer or the like. The measurement results for the kinematic viscosity may be collected by an accelerated degradation test by heating or the like. The measurement results for the kinematic viscosity may be collected by an accelerated degradation test by heating or the like.

The kinematic viscosity of the compressor oil can be obtained by the following Equation (6) using the temporal change (change rate) in the kinematic viscosity of the compressor oil.

$$\begin{matrix}[ Equation 6 ] & \\ B ( t ) = B ( 0 ) + {\int }_{0}^{{t}_{1}} b ( t ) dt & ( 6 )\end{matrix}$$

[In Equation (6), b(t) represents the change rate of the kinematic viscosity of the compressor oil at time t, B(t) represents the kinematic viscosity of the compressor oil at time t, B(0) represents the kinematic viscosity of the compressor oil in the initial state, and t1 represents the elapsed time from the initial state.]

The kinematic viscosity B(0) of the compressor oil in the initial state can be obtained by actual measurement before the compressor oil is injected into the compressor. Alternatively, actual measurement can be performed at the time of sampling the compressor oil sealed in the compressor, and the time of sampling can be set to the latest initial state.

As the arithmetic expression based on the correlation between the temporal changes in the state quantities of the environmental factors and the temporal change in the physical properties of the compressor oil, when the kinematic viscosity of the compressor oil is obtained using the pressure, temperature, and moisture content of the working medium as the environmental factors, an arithmetic expression represented by the following Equation (7) or (8) can be used.

$$\begin{matrix}[ Equation 7 ] & \\ b ( t ) = {g}_{water} ( W ( t ) ) + {g}_{pressure} ( P ( t ) ) \times {g}_{temperature} ( T ( t ) ) & ( 7 )\end{matrix}$$

$$\begin{matrix}[ Equation 8 ] & \\ b ( t ) = {g}_{water} ( W ( t ) ) \times {g}_{pressure} ( P ( t ) ) \times {g}_{temperature} ( T ( t ) ) & ( 8 )\end{matrix}$$

[In Equations (7) and (8), b(t) represents the change rate of the kinematic viscosity of the compressor oil at time t, W(t) represents the temporal change in the moisture content of the working medium at time t, P(t) represents the temporal change in the pressure of the working medium at time t, T(t) represents the temporal change in the temperature of the working medium at time t, g_{water} represents the correlation function between the moisture content of the working medium and the kinematic viscosity during the change in the compressor oil over time, gₚᵣₑₛₛᵤᵣₑ represents the correlation function between the pressure of the working medium and the kinematic viscosity during the change in the compressor oil over time, and gₜₑₘₚₑᵣₐₜᵤᵣₑ represents the correlation function between the temperature of the working medium and the kinematic viscosity during the change in the compressor oil over time.]

The correlation function g_{water} is formulated as a multivariable function that includes moisture content and kinematic viscosity as variables. The correlation function g_{water} may include a variable related to the sensor, a variable related to the compressor, a variable related to the working medium, and the like. Incorporation of such variables enables calibration of measurement errors, equipment errors, and the like.

The correlation function g_{water} can be derived from a reaction rate equation based on reaction kinetics. The reaction rate equation can be an appropriate reaction order or a combination of a plurality of reaction orders different from each other, depending on the accuracy of the prediction required and other factors.

The arithmetic expression based on the correlation represented by Equation (7) or (8) can be determined by multivariate analysis (fitting) using actual measurement results, with the temporal change in the pressure of the working medium (time derivative), the temporal change in the temperature of the working medium (time derivative), and the temporal change in the moisture content of the working medium (time derivative) as explanatory variables, and the temporal change in kinematic viscosity of the compressor oil (time derivative) as an objective variable.

As the arithmetic expression based on the correlation between the temporal changes in the state quantities of the environmental factors and the temporal change in the physical properties of the compressor oil, when the density of the compressor oil is obtained using the pressure and temperature of the working medium as the environmental factors, an arithmetic expression represented by the following Equation (9) can be used.

$$[ Equation 9 ]$$

$$c ( t ) = {h}_{pressure} ( P ( t ) ) + {h}_{temperature} ( T ( t ) ) \qquad ( 9 )$$

[In Equation (9), c(t) represents the change rate of the density of the compressor oil at time t, P(t) represents the temporal change in the pressure of the working medium at time t, T(t) represents the temporal change in the temperature of the working medium at time t, hₚᵣₑₛₛᵤᵣₑ represents the correlation function between the pressure of the working medium and the density during the change in the compressor oil over time, and hₜₑₘₚₑᵣₐₜᵤᵣₑ represents the correlation function between the temperature of the working medium and the density during the change in the compressor oil over time.]

The correlation function hₚᵣₑₛₛᵤᵣₑ is formulated as a multivariable function that includes pressure and density as variables. In addition, the correlation function hₜₑₘₚₑᵣₐₜᵤᵣₑ is formulated as a multivariable function that includes temperature and density as variables. The correlation function hₚᵣₑₛₛᵤᵣₑ and the correlation function hₜₑₘₚₑᵣₐₜᵤᵣₑ may include a variable related to the sensor, a variable related to the compressor, a variable related to the working medium, and the like. Incorporation of such variables enables calibration of measurement errors, equipment errors, and the like.

The correlation function hₚᵣₑₛₛᵤᵣₑ and the correlation function hₜₑₘₚₑᵣₐₜᵤᵣₑ can be derived from a reaction rate equation based on reaction kinetics. The reaction rate equation can be an appropriate reaction order or a combination of a plurality of reaction orders different from each other, depending on the accuracy of the prediction required and other factors. Examples of elementary reactions constituting the reaction rate equation include oxidation reactions of base oil, dissociation reactions of base oil, reactions of intermediate reaction products of base oil, and the like.

The arithmetic expression based on the correlation represented by Equation (9) can be determined by multivariate analysis (fitting) using actual measurement results, with the temporal change (time derivative) in the pressure of the working medium and the temporal change (time derivative) in the temperature of the working medium as explanatory variables and the temporal change (time derivative) in the density of the compressor oil as an objective variable.

The density of the compressor oil can be obtained by the following Equation (10) using the temporal change (change rate) in the density of the compressor oil.

$$[ Equation 10 ]$$

$$C ( t ) = C ( 0 ) + {\int }_{0}^{{t}_{1}} c ( t ) dt \qquad ( 10 )$$

[In Equation (10), c(t) represents the change rate of the density of the compressor oil at time t, C(t) represents the density of the compressor oil at time t, C(0) represents the density of the compressor oil in the initial state, and t1 represents the elapsed time from the initial state.]

The density C(0) of the compressor oil in the initial state can be obtained by actual measurement before the compressor oil is injected into the compressor. Alternatively, actual measurement can be performed at the time of sampling the compressor oil sealed in the compressor, and the time of sampling can be set to the latest initial state.

As the arithmetic expression based on the correlation between the temporal changes in the state quantities of the environmental factors and the temporal change in the physical properties of the compressor oil, when the density of the compressor oil is obtained using the pressure, temperature, and moisture content of the working medium as the environmental factors, an arithmetic expression represented by the following Equation (11) or (12) can be used.

$$[ Equation 11 ]$$

$$c ( t ) = {h}_{water} ( W ( t ) ) + {h}_{pressure} ( P ( t ) ) \times {h}_{tempeature} ( T ( t ) ) \qquad ( 11 )$$

$$[ Equation 12 ]$$

$$c ( t ) = {h}_{water} ( W ( t ) ) + {h}_{pressure} ( P ( t ) ) \times {h}_{tempeature} ( T ( t ) ) \qquad ( 12 )$$

[In Equations (11) and (12), c(t) represents the change rate of the density of the compressor oil at time t, W(t) represents the temporal change in the moisture content of the working medium at time t, P(t) represents the temporal change in the pressure of the working medium at time t, T(t) represents the temporal change in the temperature of the working medium at time t, h_{water} represents the correlation function between the moisture content of the working medium and the density during the change in the compressor oil over time, hₚᵣₑₛₛᵤᵣₑ represents the correlation function between the pressure of the working medium and the density during the change in the compressor oil over time, and hₜₑₘₚₑᵣₐₜᵤᵣₑ represents the correlation function between the temperature of the working medium and the density during the change in the compressor oil over time.]

The correlation function h_{water} is formulated as a multivariable function that includes moisture content and density as variables. The correlation function h_{water} may include a variable related to the sensor, a variable related to the compressor, a variable related to the working medium, and the like. Incorporation of such variables enables calibration of measurement errors, equipment errors, and the like.

The correlation function h_{water} can be derived from a reaction rate equation based on reaction kinetics. The reaction rate equation can be an appropriate reaction order or a combination of a plurality of reaction orders different from each other, depending on the accuracy of the prediction required and other factors.

The arithmetic expression based on the correlation represented by Equation (11) or (12) can be determined by multivariate analysis (fitting) using actual measurement results, with the temporal change in the pressure of the working medium (time derivative), the temporal change in the temperature of the working medium (time derivative), and the temporal change in the moisture content of the working medium (time derivative) as explanatory variables, and the temporal change in density of the compressor oil (time derivative) as an objective variable.

When the kinematic viscosity and the density are predicted as the physical properties of the compressor oil, the process of predicting the physical properties of the compressor oil can be performed using the pressure measurement data 210 and the temperature measurement data 220 collected by the compressor 1, the moisture measurement data 230, which is used as necessary, and the correlation data indicating the correlation represented by each of Equations (5) to (12).

The correlation represented by Equation (7) or (11) can be used when the hydrolysis reaction can be regarded as non-pressure-dependent or non-temperature-dependent, such as when the moisture content is large and excessive. The correlation represented by Equation (8) or (12) can be used when the hydrolysis reaction can be regarded as pressure-dependent or temperature-dependent.

When the kinematic viscosity and the density are predicted as the physical properties of the compressor oil, measurement data is acquired similarly to the process shown in FIG. 4 (step S10). Then, based on the measurement data, the temporal change in the pressure of the working medium, the temporal change in the temperature of the working medium, and the temporal change in the moisture content of the working medium, which is used as necessary, are calculated (step S11).

Subsequently, each module of the physical property calculation unit 12 acquires correlation data for the corresponding physical properties (step S12). Each module applies the calculated temporal changes in the pressure and temperature of the working medium, and the temporal change in the moisture content of the working medium, which is used as necessary, to the correlation represented by the arithmetic equation (5) or (9) or the correlation represented by the arithmetic equation (7), (8), (11), or (12), to calculate the temporal change in the physical properties of the compressor oil (step S13).

Subsequently, each module of the physical property calculation unit 12 reads input condition data representing the physical properties of the compressor oil in the initial state for the corresponding physical properties, and time-integrates the calculated temporal change in the physical properties of the compressor oil to calculate the predicted value of the physical properties of the compressor oil (step S14). Thereafter, the data on the calculated prediction result for the physical properties of the compressor oil is output (step S15).

The prediction results for the physical properties of the compressor oil may be displayed independently for each of the physical properties of the total acid number, the kinematic viscosity, and the density, as the result of a single process, or may be displayed collectively as the result of a plurality of processes. As the prediction result for the physical properties of the compressor oil, the calculation result for the viscosity (viscosity coefficient) of the compressor oil may be displayed based on the predicted value of the kinematic viscosity of the compressor oil and the predicted value of the density.

### Third Embodiment

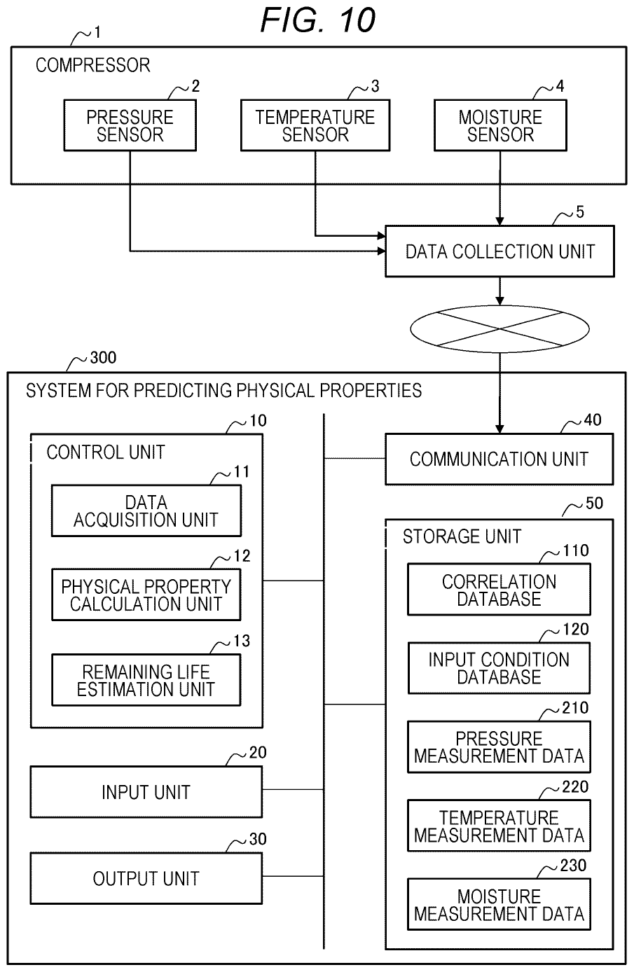

FIG. 10 is a diagram showing a configuration of a system for predicting physical properties of compressor oil according to a third embodiment.

As shown in FIG. 10, a system 300 for predicting physical properties of compressor oil according to the third embodiment includes a control unit 10, an input unit 20, an output unit 30, a communication unit 40, and a storage unit 50. The system 100 for predicting physical properties is formed of hardware such as a computer.

The system 300 for predicting physical properties is a system that predicts physical properties of compressor oil sealed in a compressor. The system 300 for predicting physical properties differs from the system 200 for predicting physical properties in having a function of estimating the remaining life of compressor oil. Other main configurations are similar to those of the system 200 for predicting physical properties. The function of estimating the remaining life of compressor oil may be provided in the system 100 for predicting physical properties.

The control unit 10 includes a remaining life estimation unit 13 in addition to the data acquisition unit 11, the physical property calculation unit 12, the control timer, and the like. The data acquisition unit 11, the physical property calculation unit 12, and the remaining life estimation unit 13 can be read as a program or a module and implemented by the control unit 10. Alternatively, the data acquisition unit 11, the physical property calculation unit 12, and the remaining life estimation unit 13 may be implemented by independent or integrated hardware modules.

The remaining life estimation unit 13 estimates the remaining life of the compressor oil based on the result of the process of predicting the physical properties of the compressor oil. The remaining life estimation unit 13 calculates the degradation rate of the compressor oil using the prediction result for the physical properties of the compressor oil calculated by the physical property calculation unit 12 as an input, and calculates the remaining life of the compressor oil based on the degradation rate of the compressor oil.

The degradation rate of the compressor oil can be obtained by the following Equation (13) using the total acid number of the compressor oil.

$$[ Equation 13 ]$$

$$R ( t ) = α \times \frac{A ( t ) - A ( 0 )}{{A}_{L} - A ( 0 )} \times 100 \qquad ( 13 )$$

[In Equation (13), R(t) represents the degradation rate of the compressor oil at time t, x represents the safety factor of the compressor, A(t) represents the total acid number of the compressor oil at time t, A(0) represents the total acid number of the compressor oil in the initial state, and A_{L} represents the total acid number of the compressor oil at the end of its service life.]

As the safety factor α of the compressor, any numerical value can be set for each compressor. The safety factor α of the compressor can be set, for example, as the ratio between the minimum value of the total acid number of the compressor oil, at which the normal function of the compressor oil cannot be obtained in the compressor, and the maximum value of the total acid number of the compressor oil, indicated by the compressor oil during normal operation. Information on the safety factor α of the compressor can be entered into the input condition database 120 of the storage unit 50.

The total acid number A(0) of the compressor oil in the initial state can be obtained by actual measurement before the compressor oil is injected into the compressor. Alternatively, actual measurement can be performed at the time of sampling the compressor oil sealed in the compressor, and the time of sampling can be set to the latest initial state.

As the total acid number A_{L} of the compressor oil at the end of its service life, any numerical value can be set for each compressor oil or each compressor. As the total acid number A_{L} of the compressor oil at the end of its service life, for example, the minimum value of the total acid number of the compressor oil, at which the normal function of the compressor oil cannot be obtained, can be set. Information on the total acid number A_{L} of the compressor oil at the end of its service life can be entered into the input condition database 120 of the storage unit 50.

The remaining life of the compressor oil can be obtained by the following Equation (14) using the degradation rate of the compressor oil.

$$[ Equation 14 ]$$

$${t}_{L} = \frac{1 - R ( {t}_{2} )}{R ( {t}_{2} )} \times {t}_{2} \qquad ( 14 )$$

[In Equation (14), T_{L} represents the remaining life of the compressor oil, R(t) represents the degradation rate of the compressor oil at time t, and t2 represents the elapsed time from the initial state.]

The elapsed time t2 can be designated for each request for estimating the remaining life of the compressor oil based on an appropriate time. The elapsed time t2 may be based on any of the following: the operating time of the compressor during which the compressor compression process is performed, the usage time of the compressor calculated from the start of the use of the compressor, the usage time of the compressor oil calculated from the start of the use of the compressor oil, or other times. The elapsed time t2 is preferably based on the operating time of the compressor from the viewpoint of evaluating the usable time of the compressor corresponding to the remaining life of the compressor oil.

Similarly to the system 200 for predicting physical properties described above, the system 300 for predicting physical properties stores a correlation database 110 and an input condition database 120 in advance of the process of predicting the physical properties of the compressor oil. The input condition database 120 is configured to include data on the total acid number of the compressor oil at the end of its service life and the data of the safety factor of the compressor.

Measurement data measured by the compressor 1 is input to the system 300 for predicting physical properties in response to a request for predicting the physical properties of the compressor oil. The pressure measurement data 210, the temperature measurement data 220, and the moisture measurement data 230 can be stored in the storage unit 50. The pressure measurement data 210, the temperature measurement data 220, and the moisture measurement data 230 are stored as data for each request for predicting the physical properties of the compressor oil, for each type of compressor oil, or for each type of compressor.

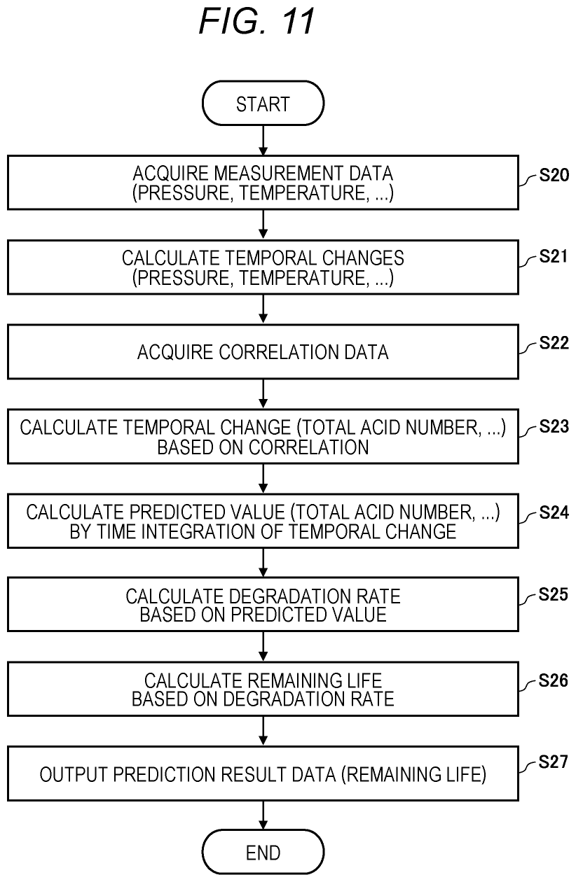

FIG. 11 is a flowchart showing the process of predicting the physical properties of the compressor oil and the process of predicting the remaining life of the compressor oil.

As shown in FIG. 11, the process of predicting the remaining life of the compressor oil can be performed using the prediction result data representing prediction results for the total acid number of the compressor oil, after the process of predicting the physical properties of the compressor oil.

When the remaining life of the compressor oil is estimated, measurement data is acquired as in the process shown in FIG. 4 (step S20). Then, based on the measurement data, the temporal change in the pressure of the working medium, the temporal change in the temperature of the working medium, and the temporal change in the moisture content of the working medium, which is used as necessary, are calculated (step S21).

Subsequently, the control unit 10 acquires correlation data for each physical property (step S22). Then, based on the correlation between the temporal changes in the state quantities of the environmental factors and the temporal change in the physical properties of the compressor oil, the temporal change in the physical properties of the compressor oil is calculated (step S23).

Subsequently, the control unit 10 reads input condition data representing the physical properties of the compressor oil in the initial state for each physical property, and the calculated temporal change in the physical properties of the compressor oil is time-integrated to calculate the predicted value of the physical properties of the compressor oil (step S24).

Subsequently, the control unit 10 calculates the degradation rate of the compressor oil based on the predicted value of the total acid number of the compressor oil (step S25). The input condition data indicating the total acid number of the compressor oil in the initial state and the input condition data indicating the total acid number of the compressor oil at the end of its service life are read from the input condition database 120 and applied to the arithmetic expression represented by Equation (13) to derive the degradation rate of the compressor oil.

Subsequently, the control unit 10 calculates the remaining life of the compressor oil based on the degradation rate of the compressor oil (step S26). The elapsed time from the initial state are referred to and applied to the arithmetic expression represented by Equation (14) to derive the remaining life of the compressor oil.

Subsequently, the control unit 10 outputs prediction result data representing the calculated predicted value of the remaining life of the compressor oil (step S15). The prediction result can be output to the output unit 30 by generating an image based on the prediction result data. As the prediction result, the predicted value of the total acid number of the compressor oil may be output together with the predicted value of the remaining life of the compressor oil.

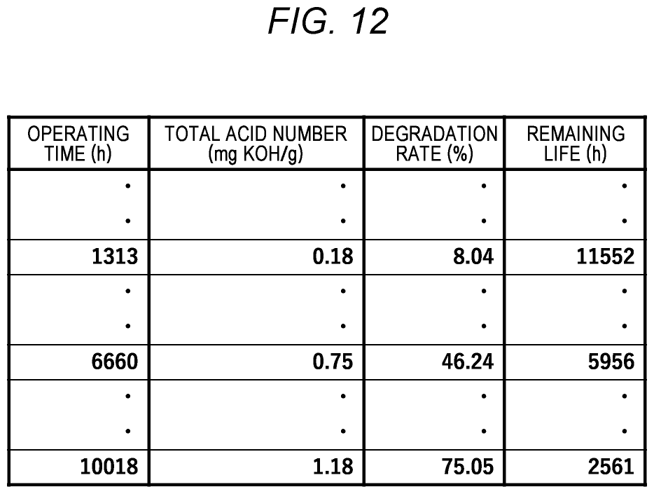

FIG. 12 is a diagram showing examples of the prediction results for the physical properties and the remaining life of the compressor oil. FIG. 12 shows the predicted value of the total acid number of the compressor oil for each operating time of the compressor predicted as the physical properties of the compressor oil, the predicted value of the degradation rate of the compressor oil for each operating time of the compressor, and the predicted value of the remaining life of the compressor oil for each operating time of the compressor.

As shown in FIG. 12, the prediction result for the remaining life of the compressor oil can be output as time-series data associated with the elapsed time. As the prediction result, the predicted value of the total acid number of the compressor oil or the predicted value of the degradation rate of the compressor oil may be output together with the predicted value of the remaining life of the compressor oil.

In FIG. 12, the prediction results for the remaining life and the like of the compressor oil are associated with the operating time of the compressor, but the prediction result for the physical properties of the compressor oil may be associated with the usage time of the compressor or the usage time of the compressor oil. The prediction result may be associated with a date and time. The operating time of the compressor, the usage time of the compressor, and the usage time of the compressor oil can each be converted into a date and time using an average elapsed time per day or similar method.

In FIG. 12, the prediction results for the remaining life and the like of the compressor oil are displayed as a table, but may be displayed as a graph, a single answer, or the like. The prediction results may be displayed independently for each request for predicting the physical properties of the compressor oil or for each mechanical unit inside the compressor, or may be displayed collectively as the result of a plurality of processes. Prediction result data representing the calculated remaining life of the compressor oil can be stored in the storage unit 50 for each request for predicting the remaining life of the compressor oil.

According to the system 300 for predicting physical properties, since the remaining life of the compressor oil is predicted based on the prediction results for the total acid number of the compressor oil, the maintenance cycle of the compressor oil can be optimized with high accuracy. The safety of the compressor can be improved because defects and accidents are less likely to occur before the next inspection of the compressor oil.

### EXAMPLE

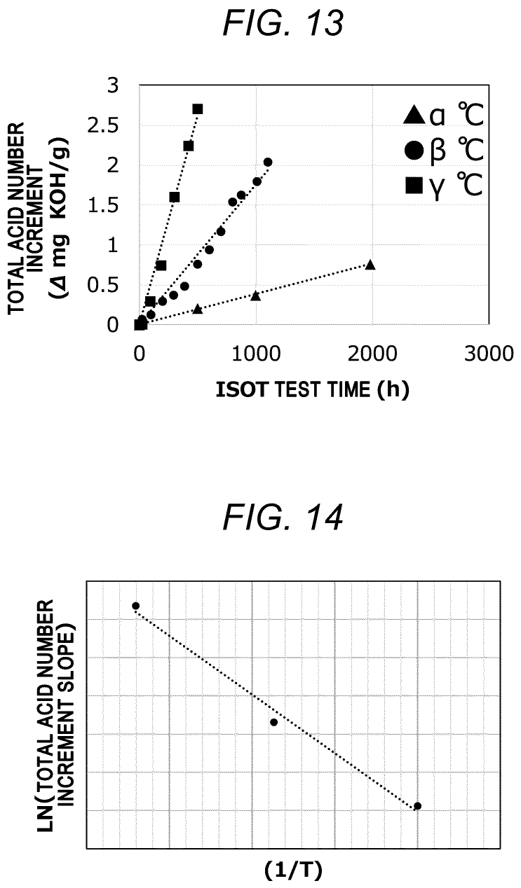

FIG. 13 is a graph showing actual measurement results for the temporal change in the total acid number of the compressor oil. FIG. 13 shows results obtained from an Indiana Stirring Oxidation Test (ISOT), a type of oxidation stability test using accelerated degradation of lubricating oil, to measure the temporal change in the total acid number of the compressor oil. In FIG. 13, the horizontal axis represents the test time [h] for performing stirring, and the vertical axis represents the increment [Δmg KOH/g] per test time of the total acid number of the compressor oil. The test was performed by adjusting an oil bath to different temperatures of α° C., β° C., and γ° C.

As shown in FIG. 13, the increment of the total acid number of the compressor oil showed linearity for each temperature. It was confirmed that the temporal change in the total acid number of the compressor oil can be approximated to a zero-order reaction with respect to the temperature.

FIG. 14 is a diagram showing Arrhenius plots of the actual measurement results for the temporal change in the total acid number. FIG. 14 shows a result of plotting the increment of the total acid number of the compressor oil measured in the ISOT test against the reciprocal of the temperature.

As shown in FIG. 14, the increment of the total acid number of the compressor oil is proportional to the inverse of the temperature. Therefore, assuming the zero-order reaction with respect to the temperature, the total acid number of the compressor oil can be estimated by the Arrhenius equation.

The total acid number of the compressor oil can be obtained by the following Equation (15) using the Arrhenius equation.

$$[ Equation 15 ]$$

$$A ( t ) = A ( 0 ) + P \times {\int }_{0}^{{t}_{1}} EXP ( - \frac{{E}_{a}}{RT ( t )} ) dt \qquad ( 15 )$$

[In Equation (15), P represents a frequency factor [mg KOH/g], Ea represents activation energy [J/mol], R represents a gas constant [J/K·mol], T(t) represents the temporal change in the temperature of the working medium at time t, A(t) represents the total acid number of the compressor oil at time t, A(0) represents the total acid number of the compressor oil in the initial state, and t1 represents the elapsed time from the initial state.]

The correlation database 110 may store data, using the Arrhenius equation as a correlation function, as correlation data representing the correlation between the temporal change in the state quantity of the environmental factor and the temporal change in the physical properties of the compressor oil. Assuming the zero-order reaction with respect to the temperature, correlation data can be constructed by collecting data corresponding to the slope and intercept of the Arrhenius plots.

Although the embodiments of the present invention have been described above, the present invention is not limited to the above embodiments, and various modifications are included as long as the modifications do not depart from the technical scope. For example, the above embodiments are not necessarily limited to those with all the configurations described above. A part of the configuration of a certain embodiment can be replaced with another configuration, or another configuration can be added to the configuration of a certain embodiment. A part of the configuration of a certain embodiment can also be subjected to the addition of another configuration, deletion, or replacement.

### REFERENCE SIGNS LIST

1 compressor
2 pressure sensor
3 temperature sensor
4 moisture sensor
5 data collection unit
10 control unit
11 data acquisition unit
12 physical property calculation unit (calculation unit)
13 remaining life estimation unit (estimation unit)
20 input unit
30 output unit
40 communication unit
50 storage unit
100 system for predicting physical properties
200 system for predicting physical properties
300 system for predicting physical properties

1. A system for predicting physical properties of compressor oil that predicts physical properties of compressor oil sealed in a compressor, the system for predicting physical properties of compressor oil comprising:
a data acquisition unit that acquires measurement data representing a temporal change in pressure of a working medium of the compressor and measurement data representing a temporal change in a temperature of the working medium of the compressor;
a calculation unit that calculates physical properties of the compressor oil sealed in the compressor, the physical properties including a total acid number, based on a correlation between the temporal change in the pressure of the working medium and the temporal change in the temperature of the working medium, and a temporal change in the physical properties of the compressor oil; and
an output unit that outputs the calculated physical properties.

2. The system for predicting physical properties of compressor oil according to claim 1, wherein
the data acquisition unit acquires the measurement data representing the temporal change in the pressure of the working medium of the compressor, the measurement data representing the temporal change in the temperature of the working medium of the compressor, and measurement data representing a temporal change in a moisture content of the working medium of the compressor, and
the calculation unit calculates the physical properties based on a correlation between the temporal change in the pressure of the working medium, the temporal change in the temperature of the working medium, and the temporal change in the moisture content of the working medium, and a temporal change in the physical properties of the compressor oil.

3. The system for predicting physical properties of compressor oil according to claim 1, wherein
the physical properties are the total acid number of the compressor oil, a kinematic viscosity of the compressor oil, and a density of the compressor oil.

4. The system for predicting physical properties of compressor oil according to claim 1, further comprising
an estimation unit that estimates a remaining life of the compressor oil based on the calculated total acid number of the compressor oil.

5. The system for predicting physical properties of compressor oil according to claim 1, wherein
the compressor is a screw compressor, a scroll compressor, a reciprocating compressor, a rotary compressor, or a turbo compressor.

6. The system for predicting physical properties of compressor oil according to claim 1, wherein
the data acquisition unit acquires the measurement data from a sensor installed in the compressor.

7. The system for predicting physical properties of compressor oil according to claim 1, further comprising
a storage unit that stores an arithmetic expression or a table that represents the correlation.

8. The system for predicting physical properties of compressor oil according to claim 1, wherein
the calculation unit obtains an increase rate of the total acid number of the compressor oil based on the following Equation (1):
$$[ Equation 1 ]$$
$$a ( t ) = {f}_{pressure} ( P ( t ) ) \times {f}_{temperature} ( T ( t ) ) \qquad ( 1 )$$
where a(t) represents the increase rate of the total acid number of the compressor oil at time t, P(t) represents the temporal change in the pressure of the working medium at time t, T(t) represents the temporal change in the temperature of the working medium at time t, fₚᵣₑₛₛᵤᵣₑ represents a correlation function between the pressure of the working medium and the total acid number during a change in the compressor oil over time, and fₜₑₘₚₑᵣₐₜᵤᵣₑ represents a correlation function between the temperature of the working medium and the total acid number during the change in the compressor oil over time.

9. The system for predicting physical properties of compressor oil according to claim 1, wherein
the calculation unit calculates the total acid number of the compressor oil based on the following Equation (2):
$$[ Equation 2 ]$$
$$A ( t ) = A ( 0 ) + {\int }_{0}^{{t}_{1}} a ( t ) dt \qquad ( 2 )$$
where a(t) represents an increase rate of the total acid number of the compressor oil at time t, A(t) represents the total acid number of the compressor oil at time t, A(0) represents the total acid number of the compressor oil in an initial state, and t1 represents an elapsed time from the initial state.

10. The system for predicting physical properties of compressor oil according to claim 2, wherein
the calculation unit obtains an increase rate of the total acid number of the compressor oil based on the following Equation (3) or (4):
$$[ Equation 3 ]$$
$$a ( t ) = {f}_{water} ( W ( t ) ) + {f}_{pressure} ( P ( t ) ) \times {f}_{tempeature} ( T ( t ) ) \qquad ( 3 )$$
$$[ Equation 4 ]$$
$$a ( t ) = {f}_{water} ( W ( t ) ) + {f}_{pressure} ( P ( t ) ) \times {f}_{tempeature} ( T ( t ) ) \qquad ( 4 )$$
where a(t) represents the increase rate of the total acid number of the compressor oil at time t, W(t) represents the temporal change in the moisture content of the working medium at time t, P(t) represents the temporal change in the pressure of the working medium at time t, T(t) represents the temporal change in the temperature of the working medium at time t, f_{water} represents a correlation function between the moisture content of the working medium and the total acid number during a change in the compressor oil over time, fₚᵣₑₛₛᵤᵣₑ represents a correlation function between the pressure of the working medium and the total acid number during the change in the compressor oil over time, and fₜₑₘₚₑᵣₐₜᵤᵣₑ represents a correlation function between the temperature of the working medium and the total acid number during the change in the compressor oil over time.

11. The system for predicting physical properties of compressor oil according to claim 1, wherein
the calculation unit obtains a change rate of a kinematic viscosity of the compressor oil based on the following Equation (5):
$$[ Equation 5 ]$$
$$b ( t ) = {g}_{pressure} ( P ( t ) ) + {g}_{temperature} ( T ( t ) ) \qquad ( 5 )$$
where b(t) represents the change rate of the kinematic viscosity of the compressor oil at time t, P(t) represents the temporal change in the pressure of the working medium at time t, T(t) represents the temporal change in the temperature of the working medium at time t, gₚᵣₑₛₛᵤᵣₑ represents a correlation function between the pressure of the working medium and the kinematic viscosity during a change in the compressor oil over time, and gₜₑₘₚₑᵣₐₜᵤᵣₑ represents a correlation function between the temperature of the working medium and the kinematic viscosity during the change in the compressor oil over time.

12. The system for predicting physical properties of compressor oil according to claim 1, wherein
the calculation unit calculates a kinematic viscosity of the compressor oil based on the following Equation (6):
$$[ Equation 6 ]$$
$$B ( t ) = B ( 0 ) + {\int }_{0}^{{t}_{1}} b ( t ) dt \qquad ( 6 )$$
where b(t) represents a change rate of the kinematic viscosity of the compressor oil at time t, B(t) represents the kinematic viscosity of the compressor oil at time t, B(0) represents the kinematic viscosity of the compressor oil in an initial state, and t1 represents an elapsed time from the initial state.

13. The system for predicting physical properties of compressor oil according to claim 2, wherein
the calculation unit obtains a change rate of a kinematic viscosity of the compressor oil based on the following Equation (7) or (8):
$$[ Equation 7 ]$$
$$b ( t ) = {g}_{water} ( W ( t ) ) + {g}_{pressure} ( P ( t ) ) \times {g}_{tempeature} ( T ( t ) ) \qquad ( 7 )$$
$$[ Equation 8 ]$$
$$b ( t ) = {g}_{water} ( W ( t ) ) + {g}_{pressure} ( P ( t ) ) \times {g}_{tempeature} ( T ( t ) ) \qquad ( 8 )$$
where b(t) represents the change rate of the kinematic viscosity of the compressor oil at time t, W(t) represents the temporal change in the moisture content of the working medium at time t, P(t) represents the temporal change in the pressure of the working medium at time t, T(t) represents the temporal change in the temperature of the working medium at time t, g_{water} represents a correlation function between the moisture content of the working medium and the kinematic viscosity during a change in the compressor oil over time, gₚᵣₑₛₛᵤᵣₑ represents a correlation function between the pressure of the working medium and the kinematic viscosity during the change in the compressor oil over time, and gₜₑₘₚₑᵣₐₜᵤᵣₑ represents a correlation function between the temperature of the working medium and the kinematic viscosity during the change in the compressor oil over time.

14. The system for predicting physical properties of compressor oil according to claim 1, wherein
the calculation unit obtains a change rate of a density of the compressor oil based on the following Equation (9):
$$[ Equation 9 ]$$
$$c ( t ) = {h}_{pressure} ( P ( t ) ) \times {h}_{temperature} ( T ( t ) ) \qquad ( 9 )$$
where c(t) represents the change rate of the density of the compressor oil at time t, P(t) represents the temporal change in the pressure of the working medium at time t, T(t) represents the temporal change in the temperature of the working medium at time t, hₚᵣₑₛₛᵤᵣₑ represents a correlation function between the pressure of the working medium and the density during a change in the compressor oil over time, and hₜₑₘₚₑᵣₐₜᵤᵣₑ represents a correlation function between the temperature of the working medium and the density during the change in the compressor oil over time.

15. The system for predicting physical properties of compressor oil according to claim 1, wherein
the calculation unit calculates a density of the compressor oil based on the following Equation (10):
$$[ Equation 10 ]$$
$$C ( t ) = C ( 0 ) + {\int }_{0}^{{t}_{1}} c ( t ) dt \qquad ( 10 )$$
where c(t) represents a change rate of the density of the compressor oil at time t, C(t) represents the density of the compressor oil at time t, C(0) represents the density of the compressor oil in an initial state, and t1 represents an elapsed time from the initial state.

16. The system for predicting physical properties of compressor oil according to claim 2, wherein
the calculation unit obtains a change rate of a density of the compressor oil based on the following Equation (11) or (12):
$$[ Equation 11 ]$$
$$c ( t ) = {h}_{water} ( W ( t ) ) \times {h}_{pressure} ( P ( t ) ) \times {h}_{temperature} ( T ( t ) ) \qquad ( 11 )$$
$$[ Equation 12 ]$$
$$c ( t ) = {h}_{water} ( W ( t ) ) \times {h}_{pressure} ( P ( t ) ) \times {h}_{temperature} ( T ( t ) ) \qquad ( 12 )$$
where c(t) represents the change rate of the density of the compressor oil at time t, W(t) represents the temporal change in the moisture content of the working medium at time t, P(t) represents the temporal change in the pressure of the working medium at time t, T(t) represents the temporal change in the temperature of the working medium at time t, h_{water} represents a correlation function between the moisture content of the working medium and the density during a change in the compressor oil over time, hₚᵣₑₛₛᵤᵣₑ represents a correlation function between the pressure of the working medium and the density during the change in the compressor oil over time, and hₜₑₘₚₑᵣₐₜᵤᵣₑ represents a correlation function between the temperature of the working medium and the density during the change in the compressor oil over time.
